(19) United States
(12) Patent Application Publication
(10) Pub. No.: US 2010/0028205 A1
(43) Pub. Date: Feb. 4, 2010

(54) MICRO-FLUIDIC DEVICE FOR THE USE IN BIOCHIPS OR BIOSYSTEMS

(75) Inventors: Marc Wilhelmus Gijsbert Ponjee, Eindhoven (NL); Jacob Marinus Jan Den Toonder, Eindhoven (NL); Murray Fulton Gilles, Eindhoven (NL); Mark Thomas Johnson, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/441,552

(22) PCT Filed: Sep. 19, 2007
(86) PCT No.: PCT/IB07/53792

§ 371 Date: Mar. 17, 2009

(30) Foreign Application Priority Data
Sep. 20, 2006 (EP) 06120932.6

(51) Int. Cl. G01N 33/48; B21D 51/16

(52) U.S. Cl. 422/68.1; 29/890.09

(57) ABSTRACT

The Invention concerns to a micro-fluidic device for the use in biochip or bio-system, wherein an 2 dimensional matrix array of temperature sensitive Poly-MEM actuators (1) is arranged in a 2 dimensional thermal processing array, in which each single temperature control element (6) or thermal element can be steered independently from each other, in order to be able to activate each single poly-MEM actuator (1).

The invention relates to a micro-fluidic device for the use in biochips or biosystems.

Micro-fluidic chips are becoming a key foundation for products in human health care, like biosensors. In all micro fluidic devices, there is a basic need to control a well definable fluid flow. Fluids must be transported, mixed, separated and directed through a micro-channel system consisting of channels with a typical width of 0.1 mm.

Various actuation mechanisms have been developed and are used. One example is disclosed in the US 2004124384 A1, where an electrostatic deformable thin film as an actuation element is shown and described. This actuation element is used as an opening and closing element of a micro valve.

Most micro actuators like this are actuated by magnetic or electric fields. An unlike disadvantage by actuation of the micro-actuators by electric or magnetic field is, that these fields, which locally have to be very high, can influence the fluids in a disturbing way, or can cause electrolyses.

So it is an object of the present invention to achieve a micro-fluidic device for the use in biochips or biosystems, in which the actuators can be steered precisely and easy, and in which disturbing effects on the fluids are avoided.

To this end a 2-dimensional matrix array of temperature sensitive polymer-MEM actuators is applied, which is arranged on a 2-dimensional thermal processing array, in which each single thermal element can be steered independently from each other, in order to be able to activate each single polymer-MEM actuator. MEM is a common abbreviation which stands for Micro ElectroMechanical.

By this the arrays, that means the thermal actuator array or matrix is folded on the 2-dimensional thermal element array or matrix. So each single actuator is stimulated by its single thermal element. The thermal elements itself can independently be steered by a matrix array steering as described in the following part. For the reason actuator stimulation is only caused by local thermal energy, each form of electrolyses or electric or magnetic influence on the fluid is avoided.

So the stated object is achieved for a micro-fluidic device for the use in biosensors by the features of patent claim 1.

Further embodiments of this system or device are characterized in the dependant claims 2-8.

The basic idea and function of the invention are, that the actuators are stimulated thermally.

Furthermore, a method to operate a micro-fluidic device is given by features of claim 9.

Further embodiments of this are given in claim 10 and 11.

In the present invention it is proposed to use polymer micro-actuators that are stimulated by a change in temperature, and that can be addressed locally on the basis of an active matrix array.

With respect to electrical-field actuation, thermal addressing of polymer micro-actuators is beneficial as the heat can be locally generated (e.g. by feeding current through a resistor) without the need for high electric fields that can disturb the motion of bio-molecules or cause electrolyses.

A first embodiment of the invention is, that the thermal processing array consists of single temperature control elements TCE. Because each polymer-MEM actuator will be actuated thermally and independent from each other, each of it needs a single and independent temperature control element.

In a further embodiment a single thermal control element TCE per column is coupled in time actuation, in order to actuate only a single thermal control element TCE per column at the same time.

In a very advantageous embodiment each thermal control element TCE is a Peltier-element. By the use of a Peltier-element it is possible to switch temperature-variations very quick and precise. Another effect by the use of Peltier-elements is, that these can be operated as heating elements as well as cooling elements. So it is possible to drive the temperature by a combined heating and cooling very precise and quick.

In this sense and in a micro-sized sense it is very advantageous, that the Peltier-element is a thin film Peltier-element.

For realizing a quick actuation a powerful steering of the elements is necessary, so that in a further embodiment a local current source is arranged at each temperature control element TCE.

In a further embodiment the local current source is a transistor wherein the mobility and/or the threshold voltage variations are at least partially compensated.

In an important embodiment thermal sensors are located at each polymer-MEM actuator, in order to control the evaluated and steered temperature. This completes the aforesaid powerful and quick steering of the temperature control elements.

Finally the precise and powerful actuation of the polymer-MEMs results in an effective micro pumping.

According to a method the basic inventive idea is that a 2-dimensional matrix array of temperature sensitive polymer-MEM actuators is arranged on a 2-dimensional thermal processing array, in which each single thermal element can be steered independently from each other, in order to be able to activate each single polymer-MEM actuator, by generating 2-dimensional coordinated signals to the TCE row-wise and column-wise independently from each other.

In a further embodiment the local temperature at the place of each polymer-MEM is measured and feed back into the operation means of the polymer-MEMs.

Finally also in the method each polymer-MEM will be steered by a located power source, in order to operate in short time actuation response.

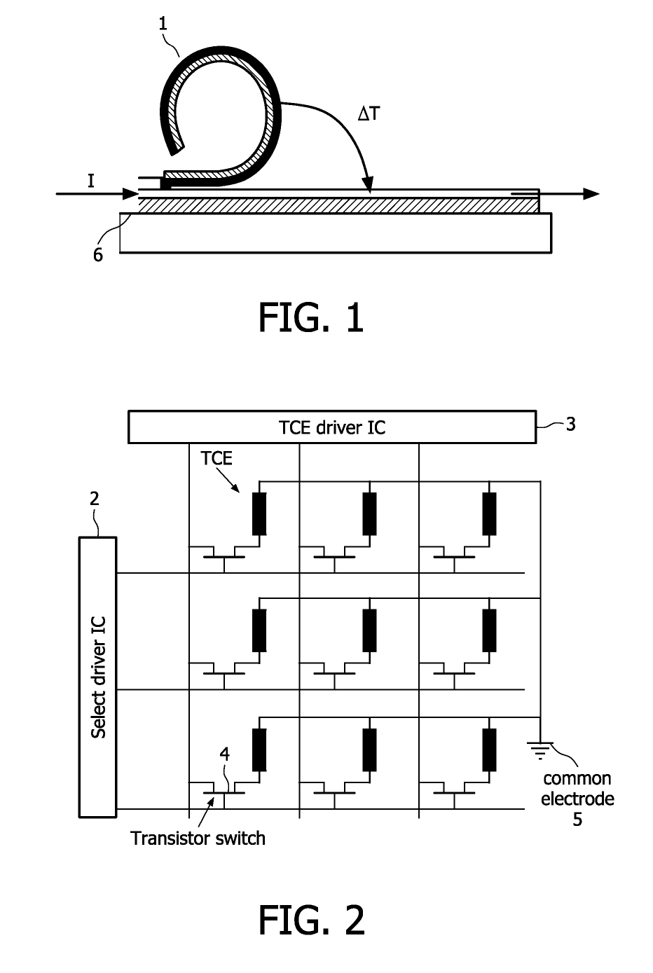

Different embodiments of the invention are shown in FIG. 1 to FIG. 6.

FIG. 1 shows the principle of rollable thermal controlled polymer-MEM

FIG. 2 shows the principle of a TCE (temperature control element)-matrix

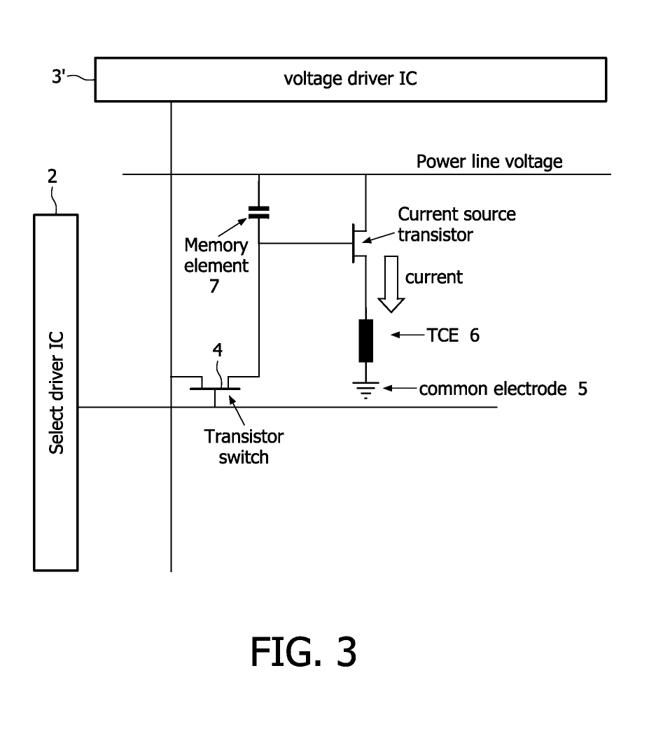

FIG. 3 shows a detailed local driver

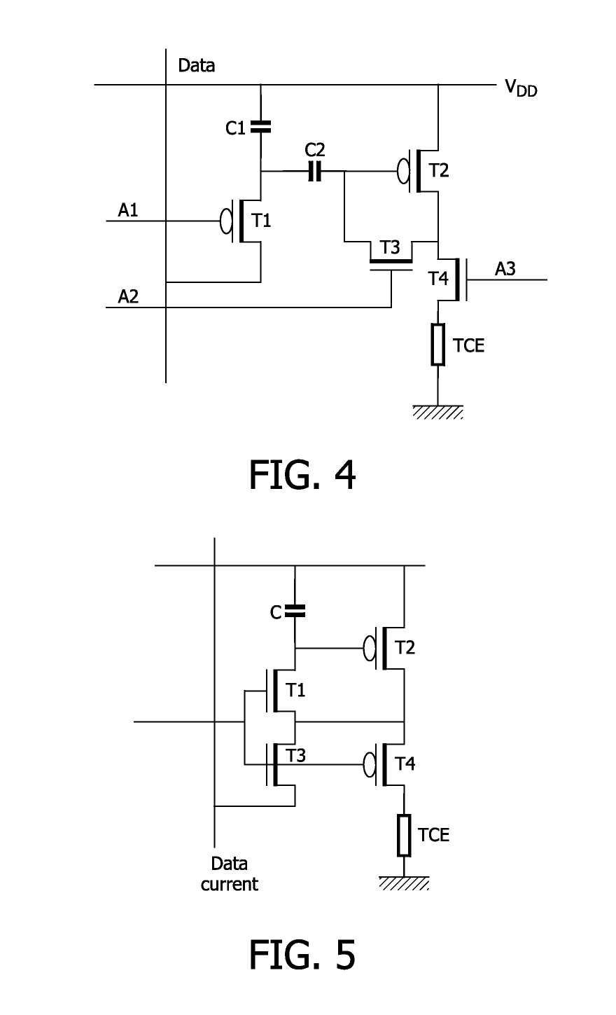

FIG. 4 shows a detailed local current source

FIG. 5 shows an alternative local current source

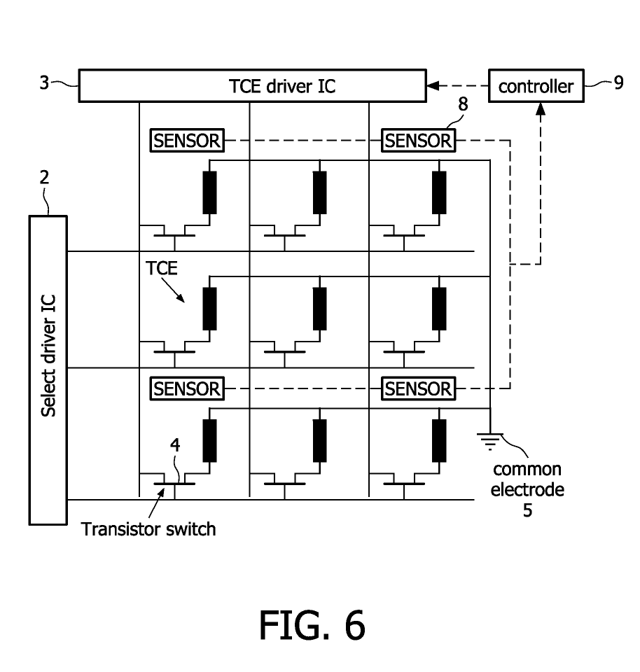

FIG. 6 shows an active TCE-matrix with local sensors

FIG. 1 shows a convenient polymer micro-actuator geometry that can be exploited. The micro-actuator or -element 1 is curled upwards in the non-actuated position. This may be caused by an internal mechanical moment that can be introduced into the film in various ways during processing. When the micro-actuator 1 is heated up by the thermal element 6 under it, above a certain critical temperature, the micro-actuator or -element 1 may curl back to straighten out. The heating element 6 can easily be activated by a simple electric current which is addressed to it. The implemented resistance causes the local thermal energy. The special way to address the heating element 6 is described further on.

The deformation of the micro-actuator may also be in the other direction, i.e. in that case the element is flattened out in the non-actuated state, and curls upward due to a change in temperature. In another way it can be activated for example with the use of Peltier-elements which can be cooled by a defined direction of electric current, and be heated by reverse electric current.

Typical sizes of the actuators 1 are between 10 and 500 micron. So it is really a micro-system which can be implemented in compact systems.

Several polymeric materials that respond to a change in temperature by deforming are known. For example by incorporating LC material into an elastomeric network a material can be made which upon heating through a specific temperature undergoes a transition in the backbone of the elastomer molecules and changes length. By careful control of processing conditions it is possible to obtain a gradient in orientation of LC molecules over the thickness of the film so that one side of the film contracts while the other expands. This creates a reversible rolling up of the film at a specific temperature. By controlling the shape of the LC molecules and the crosslink density of the elastomeric network, the ratio between the shape of the film and the temperature can be tuned.

The actuation of the polymer micro-actuators 1 in a fluid, for example a biological fluid, will induce fluid flow, i.e. fluid manipulation. To achieve efficient fluid manipulation transportation, mixing, routing, or other, it is essential that the micro-actuators, or groups of them, can be addressed individually. This would enable the creation of complex fluid flow patterns. The groups of actuators could then be actuated slightly out of phase, creating e.g. a wave-like motion of the collective actuators which would result in a transporting flow. The out-of-phase actuation of groups of actuators will result in chaotic mixing patterns, if done with proper timing. Other, specific flow patterns can be achieved as well by controlled local addressing of the actuators.

This asks for a means to control locally the temperature at the positions of individual polymer micro-actuators or groups of them, i.e. the temperature control elements (TCE) will have to be individually addressed. This description provides a solution to this requirement, using active matrix technology.

Large area electronics, and specifically active matrix technology using for example Thin Film Transistors (TFT), is commonly used in the field of flat panel displays for the drive of many display effects e.g. LCD, OLED and electrophoresis.

FIG. 2 shows an embodiment were it is proposed to control the temperature of the polymer micro-actuators by adding an array of temperature control elements, e.g. heating elements, whereby it becomes possible to generate dynamic changeable pre-defined temperature profiles across the array of polymer micro-actuators, and with that generate specified local flow patterns. A thermal control element comprises a heating element. The heating element may comprise any of known concepts for heat generation, for example but not limited to a resistive strip, Peltier-element, radio frequency heating element or radiating heating element, such as an infra-red source or diode. In general, the heating element is designed to generate a certain amount of power when a given current flows through it. Hence, by controlling the magnitude of the current passing through each of the heating elements a certain temperature profile can be created.

To enhance the temperature control, means may be provided for cooling, such as active cooling elements, e.g. thin film Peltier-elements, thermal conductive layers in thermal contact with a heat sink or cold mass or a fan. Therefore, in another embodiment the thermal control element may comprise an active cooling element.

In this embodiment, the array of temperature or thermal control elements TCE can be connected to external current sources or voltage sources using the large area electronics as a simple switch, designed to route the current or voltage from the external sources to one or more of the thermal control elements. In this example, the thermal control elements are provided as a regular array of identical units, whereby the thermal control elements are connected to the driver 2, 3 via the transistors 4 of the active matrix. The gates of the transistors are connected to a select driver 2. The select driver 2 is in all cases a standard shift register gate driver as used for an AMLCD (Active Matrix Liquid Crystal Display). The source is connected to the TCE driver 3, for example a set of voltage or current drivers. In an active matrix or a multiplexed device, it is possible to re-direct a driving signal from one driver to a multiplicity of temperature control elements, without requiring that each temperature control element is connected to the outside world, which means outside of the device, by at least two contact terminals.

Alternatively, the TCE driver IC 3 in FIG. 2 could be replaced by a single current source driver and a de-multiplexing IC to route the current to one of the column lines. These alternative is shown later.

The thermal control elements TCE, e.g. heating and/or cooling elements, are preferably located between the polymer micro-actuators and a substrate that functions as a carrier for the micro-actuators. However, the heating and optional cooling elements may also be positioned on the opposite side of the micro-actuators. Alternatively, the heating elements may also be integrated in the micro-actuators themselves, e.g. as current wires or other resistive heating structures.

External current sources are preferred, as if the source is a voltage source, the current flowing through the temperature control element, e.g. heater, and hence the temperature, is often defined by the resistance of the temperature control element. For this reason, any variations in resistance will result in differences in the temperature.

For this embodiment, the switches could be realized as transistor switches, diode switches or MIM (metal-insulator-metal) diode switches, and addressing of one or more individual temperature control elements can be carried out using the well known active matrix driving principles.

It is noted that the use of thin film Peltier-elements as TCE elements may be advantageous. Thin film Peltier-elements can on one side of the element increase the temperature, i.e. to actuate the polymer micro-actuator(s), whereas the other side of the element starts cooling the surroundings. This allows one to control polymer actuators thermally without heating/cooling the sample fluid. Temperature profiles can be generated depending on the positioning and orientation of the Peltier-elements with respect to one another.

The aforesaid alternative is shown in FIG. 3. In the matrix construction, this means that only a single temperature control element (e.g. heating/cooling) per column of the device may be actuated at the same time. This severely limits the control over the temperature profile, as the temperature profile is ideally generated by activating more than one thermal control element at the same moment, and unwanted fluctuations in the temperature cause the polymer micro-actuators to change shape and with that create a disturbance on the desired flow pattern.

In this second embodiment we propose to generate a temperature profile by controlling the magnitude of a current passing through temperature control elements using an internal current source for each thermal control element. A most simple form of a temperature control element with internal current source is shown in FIG. 3. As shown in FIG. 3, if a (LTPS) transistor were to be used as a localized current source in an active matrix array of thermal control elements as is extremely suitable in the case of an array of resistive heat generating elements, the most simple form of a current source is the trans-conductance circuit with two transistors. In this case, the output of each current source is defined by

Current=constant*mobility*(Vpower−Vtemperature−Vthreshold)²

Where Vpower is the power line voltage, Vtemperature is the programmed voltage to define the local temperature and the constant is defined by the dimensions of the transistor. Such an internal current source can be used by a single temperature control element to locally control the temperature, e.g. local heat generation, whilst to activate more than one element at a time a memory element is required in the current source circuit in order to maintain the thermal control in a period whereby further thermal control elements are being activated. Such a memory element is conveniently realized in the form of a capacitor, as also shown in FIG. 3. In this manner, any number of the thermal control generating elements in the array can be operated simultaneously at any reasonable field level, whereby extremely flexible and dynamically changeable temperature profiles can be realized. For this embodiment, the switches and local current sources could be realized as transistors, and addressing of one or more individual temperature control elements can be carried out using the well known active matrix driving principles.

One problem, however, of such a large area electronics based temperature profile generating array is that large area electronics suffer from well known non-uniformity in the performance of the active elements across the substrate. In the case of the preferred LTPS technology it is known that both the mobility and the threshold voltage (Vthreshold) of transistors varies randomly from device to device, also for devices situated close to each other.

As an example, as shown in FIG. 3, if an LTPS transistor were to be used as a localized current source based upon the trans-conductance circuit with 2 transistors, the output of each current source is defined by

Current=constant*mobility*(Vpower−Vtemperature−Vthreshold)²

For this reason, any random variations of mobility or threshold will directly result in unwanted variations in the current provided and therefore to incorrect temperature profiles. This is a particular problem, as incorrect temperature profiles can result in incorrect functioning of the polymer micro-actuators, and consequently cause undesired local flow patterns.

FIG. 3 shows a local driver for an active matrix control element system in the sense of a local current driver. In this embodiment is proposed to improve the performance of a large area electronics based programmable temperature profile generating array by increasing the uniformity and/or accuracy of the temperature profile across the array. It is achieved by creating an array of local current sources whereby the variation of the output of the current source is substantially reduced compared to that found in the trans-conductance current source described above, see FIG. 3. Specifically, it is proposed to provide local current sources where either transistor variations in the mobility, the threshold voltage, or both are compensated or partially compensated. This results in a higher uniformity in the programmed current across the array.

The thermal control array can be used to either maintain a more constant temperature profile across portions of the entire device, or alternatively to create dynamically a defined temperature profile providing that the device is also configured in the preferred form of an array. In this manner, the device can operate optimally at the required temperature profiles.

Again, in all cases, the temperature profile generating array preferably comprises a multiplicity of individually addressable and drivable thermal control elements.

In FIG. 4 a further detailed embodiment according to the voltage compensating circuit is shown. By this is a threshold compensating circuit incorporated into a localized current source for application in a programmable temperature profile generating array.

A wide variety of circuits for compensating for threshold voltage variations is available, and will be incorporated within this invention; for clarity this embodiment of the invention will be illustrated using the local current source circuit shown in FIG. 4. This circuit operates by holding a reference voltage e.g. VDD on the data line with transistors T1 and T3. Transistor T4 is pulsed which causes T2 to turn on. Upon the pulse is sent, T2 charges C2 up to the threshold voltage of T2. Then T3 is turned off storing the threshold on C2. Then the data voltage is applied and C1 is charged to this voltage. Therefore the gate-source voltage of T2 is the data voltage plus its threshold. Therefore the current, which is proportional to the gate-source voltage minus the threshold voltage squared, becomes independent of the threshold voltage of T2. Therefore a uniform current can be applied to an array of thermal control elements, e.g. the heaters. An advantage of this class of circuit is that the programming of the local current source can still be carried out with a voltage signal, as is standard in active matrix display applications.

In order to address the latter point in this embodiment, it is proposed to incorporate both a mobility and threshold voltage compensating circuit into a localized current source for application in a programmable temperature profile generating array. A wide variety of circuits for compensating for both mobility and threshold voltage variations are available; for clarity this embodiment of the invention will be illustrated using the local current source circuit shown in FIG. 5. This circuit is programmed with a current when T1 and T3 are on and T4 is off. This charges capacitor C to a voltage sufficient to pass the programmed current through T2. Then T1 and T3 are turned off to store the charge on capacitor C and T4 is turned on to pass current to the thermal control element. A compensation of both threshold and mobility variations of T2 is achieved so uniform currents can be delivered to an array of thermal control elements. An advantage of this class of circuit is that variations in the mobility of the TFT will also be compensated by the circuit.

A last embodiment is shown in FIG. 6. One issue with the approaches taken in the above set of embodiments is, that the temperature profile must be defined by the data signals.

Any unexpected variation in the characteristics of the device, or its environment, will therefore result in an incorrect temperature profile. For this reason, in this embodiment an active temperature control of thermal control elements is proposed, e.g. heaters, in an active matrix array, making use of temperature sensors 8 and any of the well known feedback schemes, see FIG. 6.

The temperature sensors 8 may be any of the known, but not limited to sensors, such as resistive sensor, p-n junction based sensor, or transistor based sensor. The feedback of the sensor function to the temperature generating elements may be done either externally to the array, using an external controller 9, or even locally if the sensor 8 is combined with the array. In a preferred embodiment, the sensor could even be realized in a technology based upon the technology used to realize the thermal control element array, such as LTPS.

In different embodiments, a sensor could be associated with every thermal control element, with a plurality of thermal control elements or alternatively a multiplicity of sensors could be associated with a single thermal control generating element. This approach provides a high degree of certainty that programmed temperature profiles are actually realized, which may assist in obtaining approval to use such devices.

So by this invention it is proposed to improve the control of polymer micro-actuators in a micro-fluidic device such as a biochip or biosystem, by the integration of an array of temperature control elements (TCE), using a large area electronics based programmable matrix approach. Preferably, the thermal control array is arranged in the form of an active matrix array, with local heating elements, i.e. current sources. Preferably, the device also comprises temperature sensing elements. Preferably, the device also comprises external or local thermal feedback circuits. In addition, the device comprises means for passive cooling (i.e. cold plate, fan, heat-sink) and/or elements for active cooling (i.e. Peltier-elements).

The device may optionally comprise additional elements such as photo-sensors, and electrodes to electrically manipulate, i.e. transport, mix, concentrate (bio)molecules, e.g. DNA, proteins, cells, or create electrical fluid flows.

The device will be able to realize a variety of dynamically changeable defined temperature profiles, such that the polymer micro-actuators can be actuated at will and specified local flow patterns can be created, e.g. to mix fluids.

### POSITION NUMBERS

1. Polymer-MEMs
2. Select driver IC
3. TCE driver IC, synonym 3′
4. Transistor switch
5. common electrode
6. TCE, temperature control element
7. Memory element
8. Sensor
9. Controller
10. Local current source
T1, T2, T3, T4 Transistor
C1, C2 Capacitor
VDD Voltage data signal

1. A micro-fluidic device for the use in a biochip or bio-system, wherein a 2-dimensional matrix array of temperature sensitive polymer-MEM actuators (1) is arranged on a 2-dimensional thermal processing array, in which each single temperature control element (6) can be steered independently from each other in order to be able to activate each single polymer-MEM actuator (1).

2. A micro-fluidic device according to claim 1, characterized in that the thermal processing array consists of single temperature control elements TCE (6).

3. A micro-fluidic device according to claim 1, characterized in that a single temperature control element TCE (6) per column is coupled in time actuation, in order to actuate only a single temperature control element TCE (6) per column at the same time.

4. A micro-fluidic device according to claim 1, characterized in that the temperature control element TCE (6) is a Peltier-element.

5. A micro-fluidic device according to claim 4, characterized in that the Peltier-element (6) is a thin film Peltier-element.

6. A micro-fluidic device according to claim 1, characterized in that a local current source (10) is arranged at each temperature control element TCE (6).

7. A micro-fluidic device according to claim 6, characterized in that the local current source (10) is a transistor wherein the mobility and/or the threshold voltage variations are at least partially compensated.

8. A micro-fluidic device according to claim 1, characterized in that thermal sensors are located at each polymer-MEM actuator (1), in order to control the evaluated and steered temperature.

9. Method wherein a 2-dimensional matrix array of temperature sensitive polymer-MEM actuators (1) is arranged on a 2-dimensional thermal processing array, in which each single thermal element can be steered independently from each other, in order to be able to activate each single polymer-MEM actuator (1), by generating 2-dimensional coordinated signals to the temperature control element TCE (6) row-wise and column-wise independently from each other.

10. Method according to claim 9, characterized in that the local temperature at the place of each polymer-MEM (1) is measured and feed back into the operation means of the polymer-MEMs (1).

11. Method according to claim 9, characterized in that each polymer-MEM (1) will be steered by a located power source in order to operate in short time actuation response.
